(19) United States
(12) Patent Application Publication
(10) Pub. No.: US 2025/0140377 A1
(43) Pub. Date: May 1, 2025

(54) NUTRIENT INFUSION CONTROL METHOD, APPARATUS, DEVICE, AND STORAGE MEDIUM

(71) Applicant: Medcaptain Medical Technology Co., Ltd., Shenzhen (CN)

(72) Inventors: Minhua LIANG, Shenzhen (CN); Xiaocheng TANG, Shenzhen (CN); Liyan WEI, Shenzhen (CN); Ling HU, Shenzhen (CN); Guangyu LIU, Shenzhen (CN)

(21) Appl. No.: 18/915,284

(22) Filed: Oct. 14, 2024

(30) Foreign Application Priority Data
Oct. 26, 2023 (CN) 202311409821.6

(51) Int. Cl. G16H 20/60; A61J 15/00; A61M 5/168

(52) U.S. Cl. CPC G16H 20/60; A61J 15/0076; A61M 5/168

(57) ABSTRACT

The present application provides a nutrient pump control method, apparatus, device and storage medium. The method includes: obtaining a feeding scheme for a target object in response to a feeding instruction of the target object, wherein the feeding scheme comprises a plurality of feeding phases, and at least one feeding parameter is different in different feeding phases; and controlling the nutrient pump to output nutrients according to the feeding scheme. The method of the present application improves the accuracy and timeliness of the feeding via the nutrient infusion.

### REFERENCE TO RELATED APPLICATIONS

The invention claims priority of Chinese Patent Application No. 202311409821.6, entitled “Nutrient Infusion Control Method, Apparatus, Device and Storage Medium” filed with the China National Intellectual Property Administration on Oct. 26, 2023, which is incorporated herein by reference in its entirety.

### TECHNICAL FIELD

This invention generally relates to the technical field of intelligent feeding, and more particularly, to a nutrient infusion control method, apparatus, device and storage medium.

### BACKGROUND ART

In clinical medicine, in order to provide nutritional support for critically ill patients, it is necessary to provide enteral nutrition infusion for patients to increase their neuroendocrine function and preserve the intestinal mucosal barrier function and immune function. Due to the existence of large individual differences in patients, such as the type of disease, the degree of impairment of gastrointestinal function, and the tolerance of enteral nutrition infusion, it is necessary to carry out different infusion methods, dosages, infusion rates, and other infusion behaviors for different patients at different times. However, at present, the infusion of enteral nutrition through nutrient infusions is highly dependent on the manual operation and adjustment of medical personnel, which has the problems of poor timeliness and low accuracy.

Therefore, how to improve the accuracy and timeliness of enteral nutrition infusion by nutrient infusion is an urgent problem to be solved.

### SUMMARY OF THE INVENTION

The present application provides a nutrient infusion control method, device, apparatus, and storage medium to solve the problem of how to improve the accuracy and timeliness of the infusion of enteral nutrition by a nutrient infusion in the prior art.

In a first aspect, the present application provides a nutrient infusion control method, comprising:
obtaining a feeding scheme for a target object in response to a feeding instruction of the target object, wherein the feeding scheme comprises a plurality of feeding phases, and at least one feeding parameter is different in different feeding phases; and
controlling the nutrient pump to output nutrients according to the feeding scheme.

In an embodiment, the plurality of feeding phases comprise at least one first feeding phase, and at least one feeding parameter of the first feeding phase is changed gradually.

In an embodiment, the plurality of feeding phases comprise at least one second feeding phase; the second feeding phase comprising a plurality of feeding sub-phases, and at least a part of the feeding sub-phases have the same or different feeding parameters.

In an embodiment, the plurality of feeding sub-phases comprise at least one target feeding sub-phase, and at least one feeding parameter of the target feeding sub-phase is changed gradually.

In an embodiment, feeding parameters of different feeding phases within a feeding cycle are changed gradually, and the feeding cycle comprises at least two feeding phases.

In an embodiment, the feeding parameter comprises at least one of the following: a feeding rate, a feeding volume, a feeding time, a feeding concentration, a feeding interval, and a duration of the feeding interval.

In an embodiment, the step of obtaining the feeding scheme for the target object comprises:
displaying a setting page of feeding parameters of the feeding phase; and
obtaining the feeding scheme for the target object in response to a setting operation of at least one feeding parameter of the feeding phase by a user.

In an embodiment, the setting page comprises a calculation strategy for calculating the feeding parameter of the feeding phase;
the step of obtaining the feeding scheme for the target object in response to the setting operation of at least one feeding parameter of the feeding phase by the user comprises:
obtaining a feeding parameter corresponding to the calculation strategy in response to a setting operation of the user for the calculation strategy; and
obtaining the feeding scheme for the target object according to the feeding parameter corresponding to the computing strategy.

In an embodiment, the method further comprises:
modifying a feeding parameter of a corresponding feeding phase to obtain a modified feeding scheme in response to a modification instruction directed to the feeding parameter of the feeding phase; and
controlling the nutrient pump to output nutrients according to the modified feeding scheme.

In a second aspect, the present application provides a nutrient infusion control device, comprising:
an obtaining module configured to obtain a feeding scheme for a target object in response to a feeding instruction of the target object, wherein the feeding scheme comprises a plurality of feeding phases, and at least one feeding parameter is different in different feeding phases; and
a control module configured to control the nutrient pump to output nutrients according to the feeding scheme.

In a third aspect, the present application provides an electronic device comprising: a processor, a communication interface, and a memory; wherein the processor is configured to be communicatively connected to the communication interface and the memory;
the memory is configured to store computer-executable instructions;
the communication interface is configured to communicate and interact with an external device; and
the processor is configured to execute the computer-executable instructions stored in the memory to implement the method as described in any one of the first aspects.

In a fourth aspect, the present application provides a computer-readable storage medium, the computer-readable storage medium storing computer-executable instructions, the computer-executable instructions when executed by the processor being configured to implement the nutrient infusion control method as described in any one of the first aspect.

In a fifth aspect, the present application provides a computer program product, the computer program product when executed by a processor being configured to implement the nutrient infusion control method as described in any one of the first aspect.

In the nutrient infusion control method, device, apparatus, and storage medium provided by the present application, a feeding scheme for a target object is obtained in response to a feeding instruction of the target object, a plurality of feeding phases of the target object and a feeding parameter corresponding to each of the feeding phases are determined from the feeding scheme, so as to control the nutrient infusion to output nutrients at a corresponding point in time according to these feeding phases and feeding parameters, which is not only capable of realizing the function of controlling the nutrient pump to perform different output effects at different feeding phases, but also reduces manual operation and avoids the problem of untimely manual operation, thereby improving the accuracy and timeliness of the feeding via the nutrient pump.

### BRIEF DESCRIPTION OF THE DRAWINGS

The accompanying drawings herein, which are incorporated into and form a part of the specification, illustrate embodiments consistent with the present application and are used in conjunction with the specification to explain the principles of the present application.

FIG. 1 shows a flowchart of a nutrient infusion control method according to embodiments of the present application.

FIG. 2 shows a schematic diagram of a gradual change of a feeding parameter of a first feeding phase according to embodiments of the present application.

FIG. 3 shows a schematic diagram of a second feeding phase according to embodiments of the present application.

FIG. 4 shows a schematic diagram of a feeding cycle according to embodiments of the present application.

FIG. 5 shows a schematic diagram of another feeding cycle according to embodiments of the present application.

FIG. 6 shows a schematic flowchart of another nutrient infusion control method according to embodiments of the present application.

FIG. 7 shows a schematic diagram of a setting page according to embodiments of the present application.

FIG. 8 shows a flowchart of another nutrient infusion control method according to embodiments of the present application.

FIG. 9 shows a schematic diagram of a control device of a nutrient infusion according to embodiments of the present application.

FIG. 10 shows a schematic structural diagram of an electronic device according to embodiments of the present application.

FIG. 11 shows a schematic structural diagram of another electronic device according to embodiments of the present application.

By means of the foregoing accompanying drawings, definite embodiments of the present application have been shown, which will be described in greater detail later. These accompanying drawings and textual descriptions are not intended to limit the scope of the present application idea in any way, but rather to illustrate the concepts of the present application for those skilled in the art by reference to particular embodiments.

### DETAILED DESCRIPTION

Example embodiments will be described herein in detail, examples of which are represented in the accompanying drawings. When the following description relates to the accompanying drawings, the same numerals in the different accompanying drawings indicate the same or similar elements unless otherwise indicated. The embodiments described in the following exemplary embodiments do not represent all embodiments consistent with the present application. Rather, they are only examples of devices and methods consistent with some aspects of the present application as detailed in the appended claims.

The nutrient infusion of the present application is used to feed nutrition to a feeding object. The feeding object may be any biological object that is dependent on nutrition and energy, for example, the feeding object may be a human patient, an animal, and the like. The nutrient is any substance that can provide energy and/or nutrition to the feeding object, for example, it may be a nutrient for providing enteral nutrition or saline.

For ease of understanding, the current feeding method is first explained as an example of providing enteral nutrition to a patient in an Intensive Care Unit (ICU).

Currently, the infusion methods of enteral nutrition include one-time push infusion, intermittent infusion, and continuous infusion via a pump, and the nutrients for the provision of such enteral nutrition are usually provided through the output of a nutrient infusion using the aforementioned infusion methods. The nutrient infusion is one of the main medical devices for clinical feeding, which can strictly control the feeding volume and speed of the nutrient, so that the nutrient can be uniformly input to the patient's body.

However, the current nutrient infusion only supports medical personnel to set feeding parameters of a single feeding output method. The medical personnel need to modify the feeding parameters of the nutrient infusion to adjust the nutrient output of the nutrient infusion. Therefore, the current feeding method requires frequent manual operation, which may lead to the problem of low accuracy and timeliness of feeding via the nutrient infusion due to untimely adjustment by the medical personnel, frequent operation prone to errors, and the like.

In view of this, in the nutrient infusion control method, device, apparatus, and storage medium provided by the present application, a feeding scheme for a target object is obtained in response to a feeding instruction of the target object, a plurality of feeding phases of the target object and a feeding parameter corresponding to each of the feeding phases are determined from the feeding scheme, so as to control the nutrient infusion to output nutrients at a corresponding point in time according to these feeding phases and feeding parameters, which is not only capable of realizing the function of controlling the nutrient pump to perform different output effects at different feeding phases, but also reduces manual operation and avoids the problem of untimely manual operation, thereby improving the accuracy and timeliness of the feeding via the nutrient pump.

The method of the present application is capable of controlling the nutrient pump to carry out gradual nutrient output in response to factors such as the patient's physical condition and tolerance level of the nutrient at different Phases. For example, the nutrient may be provided to the patient at a low concentration, low dose, and low speed when the enteral nutrition is first provided to the patient, and then the concentration, feeding rate, and feeding dose of the nutrient may be gradually increased thereafter to enable the patient to gradually adapt to the provision of the nutrient.

As an example, a target feeding dose of ¼ may be used for the first feeding, which may be incrementally increased to ½ of the target feeding dose for the second feeding and to the target feeding dose from the third feeding if the patient tolerates enteral nutrition well. For the feeding rate, for example, a lower feeding rate may be selected for the first feeding, such as a feeding rate of 25-50 ml/hour, followed by a gradual increase in the feeding rate, e.g. the second feeding adds a set increase, e.g. 25 ml/hour, to the previous feedings, until the feeding rate reaches the maximum feeding rate (e.g. 125-150 ml/hour).

The main body for performing the nutrient infusion control method provided in the present application may be a terminal device having a data processing function, or a processing chip of the terminal device, and may also be software or program code implementing the control method of the nutrient infusion. When the system is a terminal device having a data processing function, the terminal device may be a computing device such as a control device having a computing function for a nutrient infusion, or a nutrient infusion, and the computer device may be deployed with software or a program code for running the control method of the nutrient infusion, and the feeding operation of the nutrient infusion may be controlled by means of the software or the program code. The main body for performing the method may also be a cloud platform with data processing and control functions. When the executing body is a cloud platform, the method may be performed in the cloud to receive feeding instructions and issue control instructions to control the output of the nutrient infusion. The cloud platform may be logically divided into a plurality of portions according to practical needs, each portion having a different function. The portions in the cloud platform may be deployed in any two or three of the electronic devices (located on the user side), the edge environment, and the cloud environment, respectively. The edge environment is an environment that includes a collection of edge electronic devices that are close to the electronic devices, and the edge electronic devices include edge servers, edge mini-stations with computational power, and so on. Various portions of the cloud platform deployed in different environments or devices collaborate to realize the functions of data processing and control of that cloud platform. It should be understood that the present application does not make a restrictive division of which portions of the cloud platform are deployed in what specific environments, and the actual application may be adaptively deployed according to the computing power of the electronic devices, the resource occupancy of the edge environment and the cloud environment, or the specific application requirements.

In the following, for example, the control device of a nutrient pump is the main body for performing the method, the technical solution of the present application and how the technical solution of the present application solves the above technical problems are described in detail by means of specific embodiments. These following specific embodiments may be combined with each other, and the same or similar concepts or processes may not be repeated in certain embodiments. Embodiments of the present application will be described below in conjunction with the accompanying drawings.

FIG. 1 shows a flowchart of a nutrient infusion control method according to embodiments of the present application. As shown in FIG. 1, the method may include the following steps.

S101, a feeding scheme for the target object is obtained in response to a feeding instruction of the target object.

The feeding scheme includes a plurality of feeding phases, and at least one feeding parameter is different in different feeding phases. The plurality of feeding phases have intervals between which feeding is suspended, and in each of the feeding phases the feeding may be performed continuously and uninterruptedly, or may include a plurality of feeding sub-phases with feeding intervals. For example, it is assumed that the target object needs to receive continuous feeding for ten days, the nutrient infusion needs to perform three feeding outputs per day, with each feeding output being performed continuously without interruption. In this circumstance, the feeding phase may be each feeding output, or each day may be treated as a feeding phase. When each day is treated as a feeding phase, a plurality of feeding outputs with intervals may be included within that feeding phase. The feeding phases of the present application include, but are not limited to, the above exemplary manner of division, and may be determined according to practical needs, which is not limited herein.

The feeding parameter is used to control the feeding output of the nutrient infusion, and the feeding parameter may be the same or different for different feeding phases. The feeding parameter includes, but is not limited to, at least one of the following: a feeding rate, a feeding volume, a feeding time, a feeding concentration, a feeding interval, and a duration of the feeding interval.

The feeding rate is used to control the flow rate at which the nutrient pump is outputting the nutrient in performing the feeding operation, for example, the feeding rate may be controlled by changing the output flow rate of the nutrient pump, or the feeding rate may be controlled by changing the concentration of the nutrient. The feeding volume is used to control a total amount of nutrient outputted by the nutrient infusion to the target object during a feeding phase, which may be controlled by one or more of the feeding rate, the feeding time, or the feeding concentration, and the like. The feeding time is used to control a length of time that the nutrient pump outputs the nutrient to the target object within a feeding phase, and the feeding concentration is used to control a concentration of the nutrient output by the nutrient pump. The feeding interval is used to control the length of time that the nutrient pump stops carrying out the output between two feeding phases, or the length of time that it stops carrying out the output between two feeding sub-phases. The duration of feeding interval is the length of time that the output needs to stop being carried out during a feeding phase in the intermittent feeding.

The feeding instruction for the target object may be obtained by the nutrient infusion control device according to the operation of the user, for example, it may be obtained according to the instruction generated by the user by directly operating the control device, or it may be obtained by receiving the instruction from other user's personal device connected to the control device. The feeding instruction may include a feeding scheme for the target object, for example, including a feeding parameter corresponding to the feeding scheme, or an identification of the feeding scheme. The identification may be a name, a number, etc., of the feeding scheme.

If the feeding instruction includes a feeding scheme for the target object, which includes a previously stored feeding history, it is possible to extract that feeding scheme directly from that feeding instruction. If the feeding instruction includes an identification of the feeding scheme for that target object, the feeding scheme may be determined according to the identification, and a mapping relationship between the identification and the pre-stored feeding scheme in the control device.

S102, the nutrient infusion is controlled to output the nutritional product according to the feeding scheme.

The nutrient infusion control device determines at which times the nutrient infusion outputs the nutritional product and at which times it stops outputting the nutritional product, according to the feeding phases included in the feeding scheme; and determines at what one or more of a feeding rate, a feeding volume, a feeding time, a feeding concentration, a feeding interval, and the like, the nutrient infusion outputs the nutritional product during each feeding phase, according to the feeding parameters included in the feeding scheme.

As an example, there are three feeding phases included in the feeding scheme, and the feeding parameters of the three feeding phases may be shown in Table 1 below:

TABLE 1

| Feeding | Feeding | | Feeding | | Feeding | Feeding | Feeding |
|---|---|---|---|---|---|---|---|
| Phase | Time | | Interval | | Rate | Concentration | Volume |
| Phase 1 | 2 | Hour | 2 | Hour | 75 ml/h | Concentration 1 | 150 ml |
| Phase 2 | 2.5 | Hour | 1.5 | Hour | 100 ml/h | Concentration 1 | 250 ml |

| | | | | | | |
|---|---|---|---|---|---|---|
| Phase 3 | 3 | Hour | — | 125 ml/h | Concentration 2 | 375 ml |

As shown in Table 1, if the start of feeding output for Phase 1 is at 8:00, the feeding output for Phase 1 is completed at 10:00. The feeding rate, concentration, and volume of feeding for Phase 1 are shown in Table 1. After an interval of 2 hours, the feeding output of Phase 2 starts at 12:00, the feeding output of Phase 2 is completed at 14:30, and the feeding output of Phase 3 is started at 16:00 until 19:00 when the feeding scheme is concluded.

It should be understood that the above Table 1 is an example only and the present application is not limited to the feeding scheme shown in that example.

As can be seen from Table 1, the feeding parameters of each feeding phase can be set to different parameters in the present application, to achieve different variations of one or more of feeding time, feeding interval, feeding rate, feeding concentration, feeding volume, etc., without the need for the healthcare personnel to manually operate the nutrient pumps before the beginning and after the end of each feeding phase, and with only a single feeding command, the nutrient pumps can be realized to carry out the gradual change of the nutrient outputs function of the nutrient infusion.

In the nutrient infusion control method provided by the present application, a feeding scheme for a target object is obtained in response to a feeding instruction of the target object, a plurality of feeding phases of the target object and a feeding parameter corresponding to each of the feeding phases are determined from the feeding scheme, so as to control the nutrient infusion to output nutrients at a corresponding point in time according to these feeding phases and feeding parameters, which is not only capable of realizing the function of controlling the nutrient pump to perform different output effects at different feeding phases, but also reduces manual operation and avoids the problem of untimely manual operation, thereby improving the accuracy and timeliness of the feeding via the nutrient pump.

Hereinafter, a plurality of implementations of the feeding phases described in the foregoing, and the feeding parameters corresponding to such feeding phases, are described.

In the first implementation, the plurality of feeding phases includes at least one first feeding phase, and the at least one feeding parameter of the first feeding phase is changed gradually.

The first feeding phase is a continuous uninterrupted feeding, and the gradually changed feeding parameter in the first feeding phase may be one or more of the feeding rate, the feeding concentration, and the like. The gradual change means that the feeding parameter is not a fixed value in this first feeding phase, but a value that varies over time. For example, when the gradually changed feeding parameter is a feeding rate, the feeding rate in the first feeding phase may be constantly changing with a predetermined time interval, for example, it may be gradually increasing, gradually decreasing, or changing with a predetermined function, and so on. In the case of gradually increasing, for example, an increase in the feeding rate occurs every time a predetermined time interval elapses in the first feeding phase. The increase may be an increase according to a step length, which may be predetermined, or may be determined according to an initial feeding rate of the first feeding phase, a target feeding rate, and a total gradual changing duration of the first feeding phase. The total gradual changing duration is less than or equal to the total feeding duration of the first feeding phase. As an example, the initial feeding rate is 100 ml/h, the target feeding rate is 160 ml/h, and the total gradual changing duration is 60 minutes, so that the feeding rate grows by 1 ml/h per minute. The preset time interval may be set according to practical needs, which is not limited to 1 minute in this example.

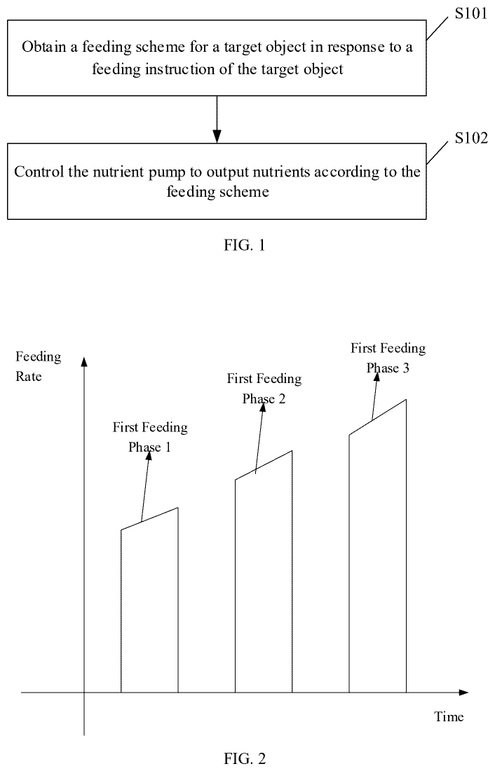

FIG. 2 shows a schematic diagram of a gradual change of a feeding parameter of a first feeding phase according to embodiments of the present application. As shown in FIG. 2, the vertical axis is the feeding rate and the horizontal axis is time, and three first feeding phases are shown in FIG. 2. The feeding rate increases asymptotically in each of the first feeding phases.

In an embodiment, the three first feeding phases may also have different gradual changing patterns, e.g. the first feeding phase 1 is gradually increasing, the first feeding phase 2 is gradually decreasing, and the first feeding phase 3 is gradually changing according to a predetermined function.

In an embodiment, the gradual changing of the three first feeding phases may be continuous or discontinuous. When the gradual changing is continuous, i.e. the initial feeding rate of each first feeding phase is the feeding rate of the last moment of the previous first feeding phase adjacent thereto. As an example, the feeding rate of the last moment of the first feeding phase 1 is the initial feeding rate of this first feeding phase 2. When the gradual changing is discontinuous, each first feeding phase may be gradually changed according to its own gradation rule, or it may be according to an average value, a median value, any one value of the previous first feeding phase as its initial feeding rate.

It should be understood that the above is only an introduction to the gradual change of the feeding parameter of the plurality of first feeding phases by taking the feeding rate as an example, and the gradual change of the feeding parameter may also be one or more other feeding parameters in the feeding interval, the feeding concentration, and the like. When the gradual changing feeding parameter is a feeding interval, the feeding interval between the plurality of first feeding phases may also be gradually changing, such as an interval of 10 minutes between the first feeding phase 1 and the first feeding phase 2, an interval of 20 minutes between the first feeding phase 2 and the first feeding phase 3, etc., in a manner similar to that of the feeding rate, which will not be repeated in the present application.

In the second implementation, the plurality of feeding phases include at least one second feeding phase. Each second feeding phase includes a plurality of feeding sub-phases, and at least a part of the feeding sub-phases has the same or different feeding parameters.

There is a feeding interval between the feeding sub-phases, i.e. the second feeding phase includes a plurality of feeding sub-phases that are independent of each other with feeding intervals. Each feeding sub-phase is continuously fed without interruption. The feeding parameters of the different feeding sub-phases may be the same or different (e.g., all the feeding parameters may be different or some of the feeding parameters may be different; or the feeding parameters of each of the feeding sub-phases may be different from the feeding parameters of the other feeding sub-phases; or the feeding parameters of some of the feeding sub-phases may be different from a part of the feeding sub-phases but the same as some of the other part of the feeding sub-phases). Alternatively, there may be a gradual change in the feeding parameters between these feeding sub-phases. The differences in the feeding parameters of the plurality of feeding sub-phases, as well as the gradual change of the feeding parameters between the feeding sub-phases are similar to the differences between the plurality of first feeding phases of the first implementation.

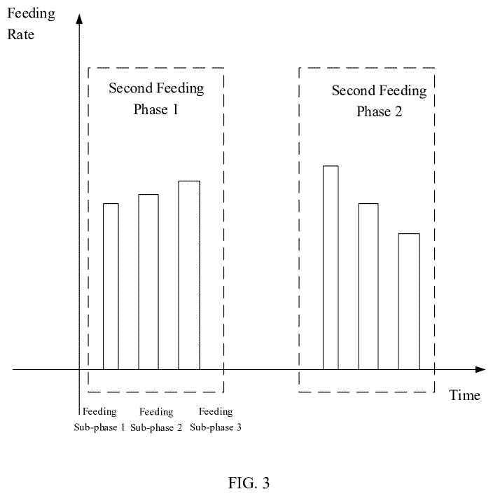

As an example, FIG. 3 shows a schematic diagram of a second feeding phase according to embodiments of the present application. As shown in FIG. 3, there are a second feeding phase 1 and a second feeding phase 2. The second feeding phase 1 includes three feeding sub-phases, each of which has a different feeding parameter. In FIG. 3, each of the feeding sub-phases has a different feeding time and a different feeding interval. As shown in FIG. 3, the feeding sub-phase 1 has a 30-minute feeding time, the feeding sub-phase 2 has a 20-minute feeding time, and the feeding sub-phase 3 has a 10-minute feeding time. The feeding interval between the feeding sub-phase 1 and the feeding sub-phase 2 is 2 minutes, and the feeding interval between the feeding sub-phase 2 and the feeding sub-phase 3 is 3 minutes.

In this example, there are feeding parameters (i.e., feeding time and feeding interval) for each feeding sub-phase in the second feeding phase 1 that are different from the feeding parameters of the other feeding sub-phases, and the other feeding parameters such as the feeding rate may be the same for all the feeding sub-phases, or partially the same, or all different.

In this implementation, further, the plurality of feeding sub-phases may include at least one target feeding sub-phase in which there is a gradual change of one or more feeding parameters. The gradual changes of the feeding parameters in the target feeding sub-phase are similar to the feeding parameter gradients in the first feeding phase in the aforementioned first implementation, which can be described with reference to the description of the gradual change of the feeding parameter in the aforementioned first feeding phase, and will not be repeated herein.

In the third implementation, the plurality of feeding phases may form a feeding cycle, and there may be a plurality of feeding cycles in a feeding scheme.

The feeding phases included in the feeding cycle may be the same feeding phase or different feeding phases. Where the feeding phases included in the feeding cycle are different feeding phases, at least one feeding parameter of these feeding phases may be gradual.

As an example, FIG. 4 shows a schematic diagram of a feeding cycle according to embodiments of the present application. As shown in FIG. 4, if the feeding scheme for the target object includes three feeding phases per day, with ten consecutive days of feeding, it may include three feeding cycles, which are feeding cycle 1, feeding cycle 2, and feeding cycle 3. The feeding phase included in the feeding cycle 1 may be a first feeding phase per day, and ten feeding phases are included in the feeding cycle 1. The feeding phase included in the feeding cycle 2 may be a second feeding phase per day, and ten feeding phases included in the feeding cycle 2. The feeding phase included in the feeding cycle 3 may be a third feeding phase per day, and ten feeding phases included in the feeding cycle 3.

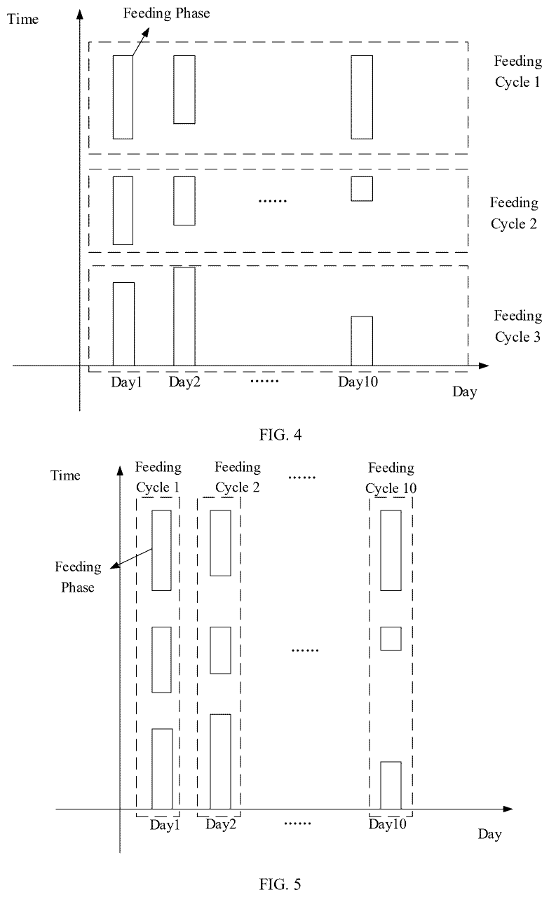

As an example, FIG. 5 shows a schematic diagram of another feeding cycle according to embodiments of the present application. As shown in FIG. 5, the feeding scheme for the target subject includes three feeding phases per day for ten consecutive days of feeding, and it may include 10 feeding cycles, which are feeding cycle 1-feeding cycle 10, each of which includes three feeding phases within a day.

In the case of the two feeding cycles described above, in some embodiments, the feeding parameters of the different feeding phases within the feeding cycle are changed gradually. The following is an example of a feeding cycle 1 shown with reference to the aforementioned FIG. 4. In the feeding cycle 1, the gradual change of the feeding parameters of each feeding phase (i.e., the first feeding phase of each day) may be, for example, one or more of the feeding rate, feeding interval, feeding time, and the like, as described previously.

In addition, the feeding parameter between such feeding cycles may also be changed gradually, for example, the feeding interval between the feeding cycle 1 and the feeding cycle 2 is different from the feeding interval between feeding cycle 2 and feeding cycle 3, thereby forming a gradual change.

It should be understood that the gradual change may also include both a gradual change in the feeding parameters of the feeding phase within the feeding cycle as described above, and a gradual change in the feeding parameters between the feeding cycles as described above.

The three implementations described above are not completely independent of each other, and the feeding solution of the present application may be a combination of some of the implementations described above, or include a combination of all of the implementations described above, which is not limited in the present application.

In the control method of the nutrient infusion provided by the embodiments of the present application, the manner in which the nutrient infusion outputs the nutrients is flexibly adjusted through different feeding parameters at different feeding phases, so as to enable the nutrient infusion to output the nutrients in a manner that supports various progressive output modes or a combination of output modes in accordance with the actual demand, thereby improving the flexibility of the nutrient infusion in outputting the nutrients, and enhancing the accuracy and timeliness of the nutrient infusion in carrying out the feeding output.

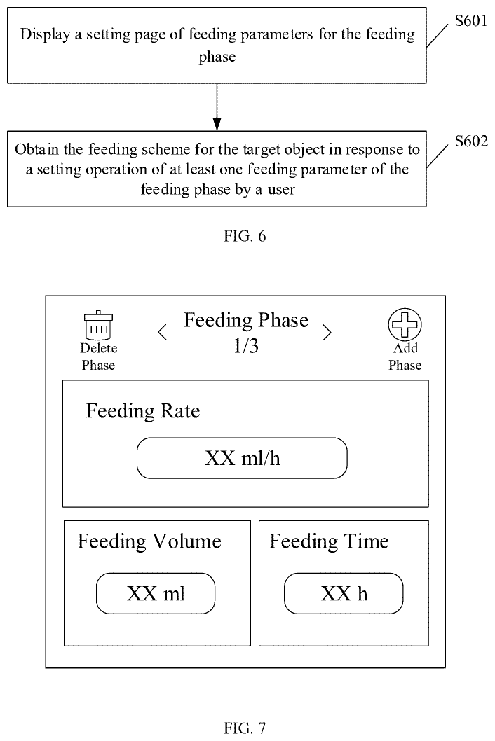

Hereinafter, a detailed description is provided for how to obtain the feeding scheme for the target object in the aforementioned step S101. FIG. 6 shows a flowchart of another control method of the nutrient infusion according to an embodiment of the present application. As shown in FIG. 6, the aforementioned step S101 may include the following steps.

S601, a setting page of feeding parameters of the feeding phase is displayed.

The setting page may include one or more feeding parameter setting sub-pages for the feeding phase, or may be a setting page for the feeding parameters of only a certain feeding phase. When the setting page is only a setting page for the feeding parameters of a certain feeding phase, the setting page may be switched to a setting page for the feeding parameters of other feeding phases by operations such as page flipping, sliding, and the like, so as to realize the setting of the feeding parameters of the plurality of feeding phases.

As an example, FIG. 7 shows a schematic diagram of a setting page provided by an embodiment of the present application. The setting page includes at least one feeding parameter and a setting area corresponding to each feeding parameter, where a value of the feeding parameter may be set to determine the feeding parameter corresponding to the feeding phase. As shown in FIG. 7, the feeding parameter may include one or more of the feeding rate, the feeding time, the feeding volume, and the like. The setting area corresponding to each feeding parameter is configured to set a value of the feeding rate, a value of the feeding time, and/or a value of the feeding volume.

The setting page may be displayed on a display screen of a nutrient infusion control device, or may be displayed on a display screen of a user's personal device connected to the control device, such as the user's computer or cell phone, which may be an interface in a web page, or an applet, or an application, to which the present application does not impose any limitation.

S602, the feeding scheme for the target object is obtained in response to a setting operation of at least one feeding parameter of the feeding phase by the user.

When the user completes the setting operation of the at least one feeding parameter of the feeding phase by entering or selecting a value of the feeding parameter through the setting page, the feeding scheme for the target object is generated according to the feeding parameters included in the page, as well as the setting value corresponding to each feeding parameter.

In some embodiments, the feeding scheme for the target object may be obtained according to the setting of the at least one feeding parameter of the plurality of feeding phases after the user has operated the plurality of feeding phases in bulk.

In the method provided by embodiments of the present application, a setting page of the feeding parameters of the feeding phases is displayed, the setting of each feeding phase and each feeding parameter are obtained according to the user's setting operation of the at least one feeding parameter of the feeding phases, and the feeding scheme for the target object is generated according to the setting, so as to facilitate the subsequent control of the nutrient pump to output nutrients according to the feeding scheme.

In a possible implementation, the setting page may further include a calculation strategy for calculating the feeding parameters of the feeding phases. The calculation strategy is used for calculating the feeding parameters of the feeding phase according to the input parameters. For example, the calculation strategy may be a calculation rule as described previously for calculating how to perform a gradual change of the feeding rate in the feeding phase according to an initial feeding rate, a target feeding rate, a preset step size, and a preset time interval.

In the implementation, the step of obtaining the feeding scheme for the target object in response to the setting operation of the user for at least one feeding parameter of the feeding phase may further include the following steps.

Feeding parameters corresponding to the calculation strategy is obtained in response to the setting operation of the user for the calculation strategy. By means of the calculation rule and the calculation parameter included in the calculation strategy, the calculation parameters are calculated in accordance with the calculation rule, so as to calculate and obtain the feeding parameters of the feeding phase. According to the feeding parameters corresponding to this calculation strategy, the feeding scheme for the target object is generated.

As an example, the calculation parameters included in the calculation strategy include an initial feeding rate, a target feeding rate, a predetermined step length, and a predetermined time interval. The calculation rule of the calculation strategy is to divide a difference between the initial feeding rate and the target feeding rate by the predetermined step length, and to determine the feeding rate each time a preset time interval elapses according to the feeding start time.

In the method provided by embodiments of the present application, a setting interface for a calculation strategy is provided to enable a user to input a corresponding calculation strategy according to the practical gradual change demand, and a gradual change feeding parameter consistent with the calculation strategy is generated according to the setting of the calculation strategy, in order to control the nutrient infusion to be able to carry out a gradual change output of nutrient according to the gradually changing feeding parameter, thereby improving the flexibility of controlling the output of nutrient from the nutrient infusion.

In addition, since the physical condition of the patient is changing at any time with the input of the nutrient, the patient may face a situation that requires the healthcare personnel to temporarily adjust the feeding parameter of the nutrient infusion. Therefore, the method of the present application may also include how to make modifications to the feeding parameters during the feeding phase.

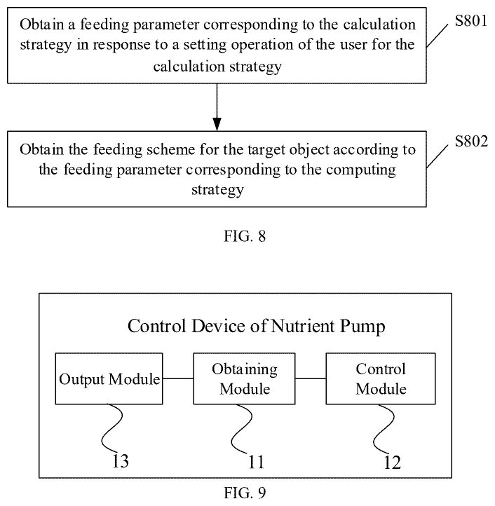

In some embodiments, FIG. 8 shows flowchart of another nutrient infusion control method according to embodiments of the present application. The control method in FIG. 8 is for adjusting a subsequent output of the nutrient infusion according to practical needs during the process of the nutrient infusion outputting nutrients according to the above-mentioned feeding scheme. As shown in FIG. 8, the method may further include the following steps.

S801, in response to a modification instruction directed to a feeding parameter of the feeding phase, the feeding parameter of the corresponding feeding phase is modified to obtain the modified feeding scheme.

The modification instruction may include an identification of the feeding phase to be modified and an identification of the feeding parameter, or may include a feeding parameter in the feeding phase to be modified and a set value of the feeding parameter.

According to the modification instruction, an operation of adding, deleting, or modifying a feeding phase, and/or an operation of modifying at least one feeding parameter, may be performed. According to the modified feeding phase and the modified feeding parameter in the feeding phase, a modified feeding scheme is generated.

S802, the nutrient pump is controlled to output the nutrient according to the modified feeding scheme.

The control device controls the output of the nutrient infusion according to the feeding phases included in the modified feeding scheme and the feeding parameters of each feeding phase, so as to control the nutrient infusion to output the nutrient to feed the patient according to the feeding phases and the feeding parameters of each feeding phase included in the modified feeding scheme.

In the method provided by embodiments of the present application, in response to a modification instruction directed to the feeding parameters of the feeding phase, the feeding parameters of the corresponding feeding phase is modified to obtain the modified feeding scheme, and the nutrient infusion is controlled to output the nutrient according to the modified feeding scheme, so as to make it easy for the user to adjust the subsequent feeding operation of the nutrient infusion after the physical condition of the patient changes with the input of the nutrient, so as to further improve the flexibility of the nutrient infusion.

FIG. 9 shows a schematic diagram of a control device of a nutrient infusion according to embodiments of the present application. As shown in FIG. 9, the nutrient infusion control device may include an obtaining module 11, a control module 12. In a possible implementation, the device may further include an output module 13.

The obtaining module 11 is configured to obtain a feeding scheme for a target object in response to a feeding instruction of the target object. The feeding scheme includes a plurality of feeding phases, and at least one feeding parameter of different feeding phases is different. In some embodiments, the feeding parameter includes at least one of the following: a feeding rate, a feeding volume, a feeding time, a feeding concentration, a feeding interval, and a feeding interval.

The control module 12 is configured to control the nutrient pump to output the nutrient according to the feeding scheme.

In some embodiments, the plurality of feeding phases includes at least one first feeding phase, and at least one feeding parameter of the first feeding phase is changed gradually. Alternatively, the plurality of feeding phases includes at least one second feeding phase. The second feeding phase includes a plurality of feeding sub-phases, and at least some of the feeding sub-phases have the same or different feeding parameters. In some embodiments, the plurality of feeding sub-phases includes at least one target feeding sub-phase, and at least one feeding parameter of the target feeding sub-phase is changed gradually. In some embodiment, the feeding scheme further includes a feeding cycle, and feeding parameters of the different feeding phases of the feeding cycle are changed gradually. The feeding cycle includes at least two such feeding phases.

In any of the above implementations, the output module 13 is configured to display a setting page of the feeding parameters of the feeding phase. The obtaining module 11 is further configured to obtain the feeding scheme for the target object in response to a setting operation of at least one feeding parameter of the feeding phase by a user.

In some embodiments, the setting page includes a calculation strategy for calculating the feeding parameters of the feeding phase. The obtaining module 11 is further configured to obtain a feeding parameter corresponding to the calculation strategy in response to a setting operation of the user for the calculation strategy, and obtain the feeding scheme for the target object according to the feeding parameter corresponding to the computing strategy. In some embodiments, the obtaining module 11 is further configured to modify a feeding parameter of a corresponding feeding phase to obtain a modified feeding scheme in response to a modification instruction directed to the feeding parameter of the feeding phase. The control module 12 is further configured to control the nutrient pump to output nutrients according to the modified feeding scheme.

The nutrient infusion control device provided in the embodiments of the present application can perform the control method of the nutrient infusion in the above method embodiments, and the realization principle and technical effect thereof are similar and will not be repeated herein.

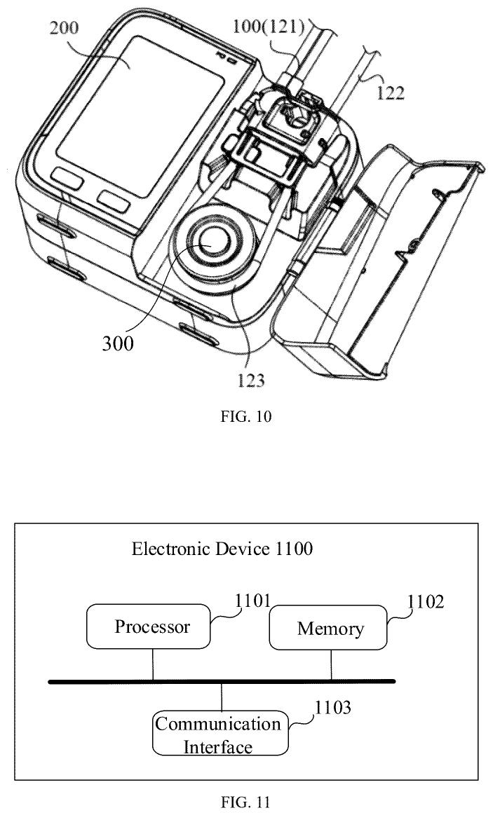

FIG. 10 shows a schematic structural diagram of an electronic device according to embodiments of the present application. The electronic device is configured to perform the control method of the nutrient infusion described in the foregoing, for example, it may be a nutrient infusion control device described in the foregoing, such as a third-party terminal, a transit station, or it may be the nutrient infusion.

As shown in FIG. 10, the electronic device is a nutrient infusion, which may include a processor, a memory, an infusion consumable 100, a touch display 200, and a drive member 300. The infusion consumable 100 may include an inlet end 121, an outlet end 122, and a silicone hose 123. The processor and the memory are disposed within the electronic device, which are not shown in the figures limited by the view angle.

When the control method of the nutrient infusion provided in the above embodiments is practiced in this electronic device, the nutritional product is input from the inlet end 121, and the processor controls the drive member 300 to squeeze the silicone hose 123 and output the nutritional product from the outlet end 122.

FIG. 11 shows a schematic structural diagram of another electronic device according to embodiments of the present application. FIG. 11 describes the internal structure of the electronic device. As shown in FIG. 11, the electronic device 1100 may include at least one processor 1101, a memory 1102, and a communication interface 1103.

The memory 1102 is configured to store a program. In some embodiments, the program may include a program code, which includes computer-executable instructions.

The memory 1102 may include a high-speed RAM memory, or may also include a non-volatile memory, such as at least one disk memory.

The processor 1101 is configured to execute the computer-executable instructions stored in the memory 1102 to implement the methods described in the preceding method embodiments. The processor 1101 may be a CPU, or an Application Specific Integrated Circuit (ASIC), or one or more integrated circuits configured to implement embodiments of the present application.

The processor 1101 may be configured to communicate and interact with an external device through the communication interface 1103, and the external device may be a personal device of a user as described previously. In a specific implementation, if the communication interface 1103, the memory 1102, and the processor 1101 are implemented independently, the communication interface 1103, the memory 1102, and the processor 1101 may be connected to each other and accomplish communication with each other via a bus. The bus may be an Industry Standard Architecture (ISA) bus, a Peripheral Component (PCI) bus, or an Extended Industry Standard Architecture (EISA) bus. The buses may be categorized as address buses, data buses, and control buses, but it does not mean that there is only one bus or one type of bus.

In some embodiments, in terms of specific implementation, if the communication interface 1103, the memory 1102, and the processor 1101 are integrated and implemented on a single chip, the communication interface 1103, the memory 1102, and the processor 1101 may complete the communication through an internal interface.

The present application also provides a computer-readable storage medium, which may include a USB flash drive, a removable hard disk, a read-only memory (ROM), a random-access memory (RAM), a magnetic disk, a CD-ROM and other kinds of media that can store program code. In an embodiment, the computer readable storage medium has program instructions stored therein, and the program instructions are used for the method in the above embodiment.

The present application also provides a program product that includes computer-executable instructions, which are stored in the readable storage medium. At least one processor of the computer device may read the computer-executable instructions from the readable storage medium, and the computer-executable instructions are executed by the at least one processor to cause the computer device to implement any one of the nutrient pump control method.

Finally, it should be noted that the above embodiments are only used to illustrate the technical solution of the present application, rather than limit the present application. Although the present application is described in detail with reference to the foregoing embodiments, a person of ordinary skill in the art should understand that it is still possible to make modifications to the technical solution documented in the foregoing embodiments, or to make equivalent substitutions for some or all of the technical features therein. The modifications or substitutions do not take the essence of the corresponding technical solutions out of the scope of the technical solutions of the embodiments of the present application.

1. A nutrient infusion control method, comprising:
obtaining a feeding scheme for a target object in response to a feeding instruction of the target object, wherein the feeding scheme comprises a plurality of feeding phases, and at least one feeding parameter is different in different feeding phases; and
controlling the nutrient pump to output nutrients according to the feeding scheme.

2. The method of claim 1, wherein the plurality of feeding phases comprise at least one first feeding phase, and at least one feeding parameter of the first feeding phase is changed gradually.

3. The method of claim 1, wherein the plurality of feeding phases comprise at least one second feeding phase;
the second feeding phase comprises a plurality of feeding sub-phases, and at least a part of the feeding sub-phases has the same or different feeding parameters.

4. The method of claim 3, wherein the plurality of feeding sub-phases comprise at least one target feeding sub-phase, and at least one feeding parameter of the target feeding sub-phase is changed gradually.

5. The method of claim 1, wherein feeding parameters of different feeding phases within a feeding cycle are changed gradually, and the feeding cycle comprises at least two of the plurality of feeding phases.

6. The method of claim 1, wherein the feeding parameter comprises at least one of the following: a feeding rate, a feeding volume, a feeding time, a feeding concentration, a feeding interval, and a duration of the feeding interval.

7. The method of claim 1, wherein the step of obtaining the feeding scheme for the target object comprises:
displaying a setting page of feeding parameters of the feeding phase; and
obtaining the feeding scheme for the target object in response to a setting operation of at least one feeding parameter of the feeding phase by a user.

8. The method of claim 7, wherein the setting page comprises a calculation strategy for calculating the feeding parameters of the feeding phase;
the step of obtaining the feeding scheme for the target object in response to the setting operation of at least one feeding parameter of the feeding phase by the user comprises:
obtaining a feeding parameter corresponding to the calculation strategy in response to a setting operation of the user for the calculation strategy; and
obtaining the feeding scheme for the target object according to the feeding parameter corresponding to the computing strategy.

9. The method of claim 7, further comprising:
modifying a feeding parameter of a corresponding feeding phase to obtain a modified feeding scheme in response to a modification instruction directed to the feeding parameter of the feeding phase; and
controlling the nutrient pump to output nutrients according to the modified feeding scheme.

10. A nutrient infusion control device, comprising:
an obtaining module configured to obtain a feeding scheme for a target object in response to a feeding instruction of the target object, wherein the feeding scheme comprises a plurality of feeding phases, and at least one feeding parameter of different feeding phases are different; and
a control module configured to control the nutrient pump to output nutrients according to the feeding scheme.

11. An electronic device, comprising: a processor, a communication interface, and a memory; wherein the processor is configured to be communicatively connected to the communication interface and the memory;
the memory is configured to store computer-executable instructions;
the communication interface is configured to communicate and interact with an external device; and
the processor is configured to execute the computer-executable instructions stored in the memory to: obtain a feeding scheme for a target object in response to a feeding instruction of the target object, wherein the feeding scheme comprises a plurality of feeding phases, and at least one feeding parameter is different in different feeding phases; and
control the nutrient pump to output nutrients according to the feeding scheme.

12. The electronic device of claim 11, wherein the plurality of feeding phases comprise at least one first feeding phase, and at least one feeding parameter of the first feeding phase is changed gradually.

13. The electronic device of claim 11, wherein the plurality of feeding phases comprise at least one second feeding phase;
the second feeding phase comprises a plurality of feeding sub-phases, and at least a part of the feeding sub-phases has the same or different feeding parameters.

14. The electronic device of claim 13, wherein the plurality of feeding sub-phase comprises at least one target feeding sub-phase, and at least one feeding parameter of the target feeding sub-phase is changed gradually.

15. The electronic device of claim 11, wherein feeding parameters of different feeding phases within a feeding cycle are changed gradually, and the feeding cycle comprises at least two of the plurality of feeding phases.

16. The electronic device of claim 11, wherein the feeding parameter comprises at least one of the following: a feeding rate, a feeding volume, a feeding time, a feeding concentration, a feeding interval, and a duration of the feeding interval.

17. The electronic device of claim 11, wherein the processor obtains the feeding scheme for the target object by:
displaying a setting page of feeding parameters of the feeding phase; and
obtaining the feeding scheme for the target object in response to a setting operation of at least one feeding parameter of the feeding phase by a user.

18. The electronic device of claim 17, wherein the setting page comprises a calculation strategy for calculating the feeding parameter of the feeding phase;
the processor obtains the feeding scheme for the target object in response to the setting operation of at least one feeding parameter of the feeding phase by the user by:
obtaining a feeding parameter corresponding to the calculation strategy in response to a setting operation of the user for the calculation strategy; and
obtaining the feeding scheme for the target object according to the feeding parameter corresponding to the computing strategy.

19. The electronic device of claim 17, wherein the processor is further configured to:
modify a feeding parameter of a corresponding feeding phase to obtain a modified feeding scheme in response to a modification instruction directed to the feeding parameter of the feeding phase; and
control the nutrient pump to output nutrients according to the modified feeding scheme.
